(19) United States
(12) Patent Application Publication
(10) Pub. No.: US 2024/0100019 A1
(43) Pub. Date: Mar. 28, 2024

(54) COMPOSITIONS COMPRISING A THERMOREVERSIBLE HYDROGEL, AND HAVING AN EXTENDED IN-USE PERIOD

(71) Applicant: UroGen Pharma Ltd., Ra'anana (IL)

(72) Inventors: Marina Konorty, Hertzlya (IL); Omer Tsipori, Shaár Efraim (IL)

(21) Appl. No.: 18/374,645

(22) Filed: Sep. 28, 2023

Related U.S. Application Data
(60) Provisional application No. 63/411,024, filed on Sep. 28, 2022.

(51) Int. Cl. A61K 31/407; A61K 9/06; A61K 47/34; A61K 47/38

(52) U.S. Cl. CPC A61K 31/407; A61K 9/06; A61K 47/34; A61K 47/38

(57) ABSTRACT

Provided herein are compositions including a thermoreversible hydrogel, and having an extended, medically suitable in-use period. A medically suitable in-use period following reconstitution can be up to about 96 hours, up to about 72 hours, up to about 48 hours, up to about 24 hours, or up to about 12 hours at room temperature. Also provided, are methods including administering the compositions up to about 96 hours, up to about 72 hours, up to about 48 hours, up to about 24 hours, or up to about 12 hours at room temperature following reconstitution.

### REFERENCE TO RELATED APPLICATIONS

This application claims priority from U.S. Provisional Patent Application No. 63/411,024, filed, Sep. 28, 2022, which is hereby incorporated by reference in its entirety.

### FIELD

The invention relates in general to compositions comprising a thermoreversible hydrogel and a therapeutic agent, such as mitomycin C, which is preferably used in treating cancer in the upper urinary tract such as, for example, cancer of the bladder and cancer of the kidney. The compositions have an extended in-use period that allows for increased efficiencies in terms of providing patient treatments, reducing pharmacy preparation time and activities, and the distribution and availability of the composition. The invention also relates to the use of these compositions in the treatment of internal body cavities by embedding a therapeutic agent in a slowly-dissolving biocompatible mixture that is applied to a surface of affected tissues. The methods are useful for the treatment of certain types of cancers, especially those of the upper urinary tract, including cancer of the bladder and/or kidneys.

### BACKGROUND

Compositions and methods of treating upper urinary tract cancers have been developed but are not optimum because they have limited in-use periods. Thus, there has been a need in the field for developing compositions and methods offering extended in-use periods.

### SUMMARY

An exemplary embodiment includes a reconstituted pharmaceutical composition, comprising a thermoreversible hydrogel and a therapeutically-effective amount of a therapeutic agent, wherein: the reconstituted pharmaceutical composition is formed by combining a first composition comprising mitomycin and a second composition comprising a thermoreversible hydrogel, and storing the reconstituted pharmaceutical composition, protected from light, at about 20° C. to about 25° C. (about 68° F. to about 77° F.) for about 8 hours to about 96 hours, wherein the reconstituted pharmaceutical composition is formulated, and remains medically-suitable, for administration to an internal body cavity of a subject; and the reconstituted pharmaceutical composition is characterized by meeting the following critical quality attributes (CQAs):
a. maintains potency of the therapeutic agent,
b. does not produce significant quantities of degradation impurities during hold time,
c. does not experience a material change in its the appearance, pH, viscosity, and/or dissolution, and/or
d. does not contain an increased risk of microbiological exposure, as indicated by a microbial challenge study and/or a microbial growth risk evaluation.

### BRIEF DESCRIPTION OF THE FIGURES

FIG. 1 shows the amount of degradation of mitomycin in NaCl solutions at different pHs and temperatures.

FIGS. 2a to 2d show Instructions for the Preparation of JELMYTO® by a pharmacy.

### DETAILED DESCRIPTION

Mitomycins are a family of aziridine-containing natural products isolated from Streptomyces caespitosus or Streptomyces lavendulae. They include mitomycin A, mitomycin B, and mitomycin C. Mitomycin C was initially discovered in the late 1950's and was determined to have activity as an antibiotic. Since then, it has been shown to have antineoplastic properties and has been used in various cancer treatments. Mitomycin is also sometimes used to treat anal cancer, cervical cancer, a type of lung cancer (non-small cell lung cancer; NSCLC), and malignant mesothelioma (cancer in the lining of the chest or abdomen) and is also sometimes used intravesically (infused directly into the bladder) to treat bladder cancer.

JELMYTO® (mitomycin) for pyelocalyceal solution is a mitomycin-containing reverse thermal gel indicated for primary chemoablative treatment of Low-Grade Upper Tract Urothelial Cancer (LG UTUC) in adults. JELMYTO® is efficacious as a primary chemoablative therapy in patients with LG UTUC. Upper Tract Urothelial Cancer (UTUC) is cancer of the lining of the bladder and other parts of the urinary tract. LG UTUC is a rare disease managed by endoscopic methods and radical nephroureterectomy. While endoscopic resection and laser ablation attempt to preserve the kidney, there is a high risk of recurrence that may eventually necessitate removal of the kidney. Although kidney removal is the gold standard for treatment of high-grade UTUC, it may be over-treatment in LG UTUC, as kidney removal offers similar five-year survival as kidney-sparing procedures but is associated with significant morbidity.

JELMYTO® is recommended for primary treatment of biopsy-proven LG UTUC in patients deemed appropriate candidates for renal-sparing therapy.

JELMYTO® is available in a single dose kit containing two 40 mg vials of mitomycin that are used for preparing solution of the therapeutic agent and one 20 mL vial of a sterile hydrogel. The product is reconstituted into an admixture by a pharmacist in accordance with the Instructions for Pharmacy and dispensed on an individual patient basis. The admixture is a viscous liquid when cooled and becomes a semi-solid gel at body temperature. The original in-use holding period of the admixture was only 8 hours at 20° C. to 25° C. (68° F. to 77° F.), which limited the availability of JELMYTO®. At the time of the development of JELMYTO®, it was unexpected that a longer holding period, such as up to 96 hours could be obtained from such a formulation. This was, in part, because it was expected that increased impurities would not be acceptable and subject to regulatory approval.

The drug slowly dissolves over four to six hours after instillation and is removed from the urinary tract by normal urine flow and voiding. The delivery system allows the initially cooled liquid to coat, fill and conform to the upper urinary tract anatomy. The cooled liquid becomes a semisolid gel at body temperature and this change in phase allows for chemoablative therapy to remain in the collecting system for four to six hours without immediately being diluted or washed away by urine flow. Chemoablation therapy uses a chemical agent to destroy (ablate) abnormal tissue or tumors. It is a minimally invasive procedure, meaning that it may be done without open surgery. Chemoablation removes a layer or layers of tissue, unlike a surgical resection, which removes an entire organ or part of it. JELMYTO® can be recommended up to a total of 12 monthly doses. To prepare JELMYTO® for administration to patients, a specific process using specialized equipment is followed. JELMYTO® is prepared in USP 800-compliant pharmacies that use heightened quality and safety standards and, in addition to compounding rooms, they can have expanded capabilities and expertise to process cytotoxic drug components. The requirements for pharmacies to be USP 800 compliant became effective Dec. 1, 2019, with compliance. During the initial allowance of JELMYTO® by the U.S. FDA, it was required that the solution used to treat a patient be discarded 8 hours after the components were combined. The material that is administered to the patient is usually prepared by a specialty pharmacy and then shipped to doctor's offices or treatment centers where it is administered to patients. Given the large geographical range of patients, combined with the narrow window (8 hours or less) between the end of preparation of the composition and the actual time when the patient is treated, a large obstacle existed between the availability of the therapeutic agent and the treatment of the patients.

In many cases, this resulted in pharmacy providers preparing the compositions in early morning hours and then having to ship the material to doctor's offices or treatment centers where it was then administered to patients within 8 hours or less of preparation. This meant that in many cases, treatments could not be performed in the morning. This required process also resulted in the inability of cancer patients located in remote locations to have access to this treatment merely due to the combination of the 8-hour window within which the composition was allowed to be administered and the logistics involved in transporting the composition to the treatment facility. In addition, a burden was placed on the shipping service, especially since it was a high priority, low size and low volume shipment and this could result in a significant increase in costs. A variety of other factors, such as weather, shipping logistics, etc. could affect the distribution of the time sensitive material.

When JELMYTO® was developed, the inventors did not expect that an extended use time for the administration of the material could be developed and approved. At the time of the initial FDA approval, the inventors did not expect that an extended use period composition that met all of the critical quality attributes (CQAs) required for an extended use composition could be developed and approved. For example, Briot et al. determined that mitomycin C (MMC) was only stable (relative concentrations systematically over 90% of the initial concentrations) for 8 hours in water for injections at a concentration of 1 mg/mL, and 10 hours in a 0.2 mg/ml 0.9% sodium chloride solution. After 96 h, however, the relative MMC concentrations were found to have dropped to below 80% as compared to initial concentrations, thus indicating significant and undesirable instability of these solutions. Briot noted that degradation products were observed and remained below 3%. Briot, Thomas, Truffaut, Christine, Le Quay, Luc, Lebreton, Anne and Lagarce, Frederic. “Stability of Reconstituted and Diluted Mitomycin C Solutions in Polypropylene Syringes and Glass Vials ” Pharmaceutical Technology in Hospital Pharmacy, vol. 1, no. 2, 2016, pp. 83-89. https://doi.org/10.1515/pthp-2016-0012 (See also https://www.degruyter.com/document/doi/10.1515/pthp-2016-0012/html?lang=en)

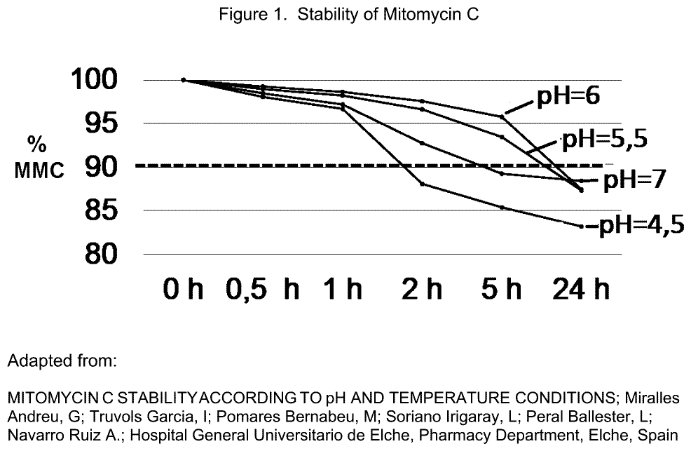

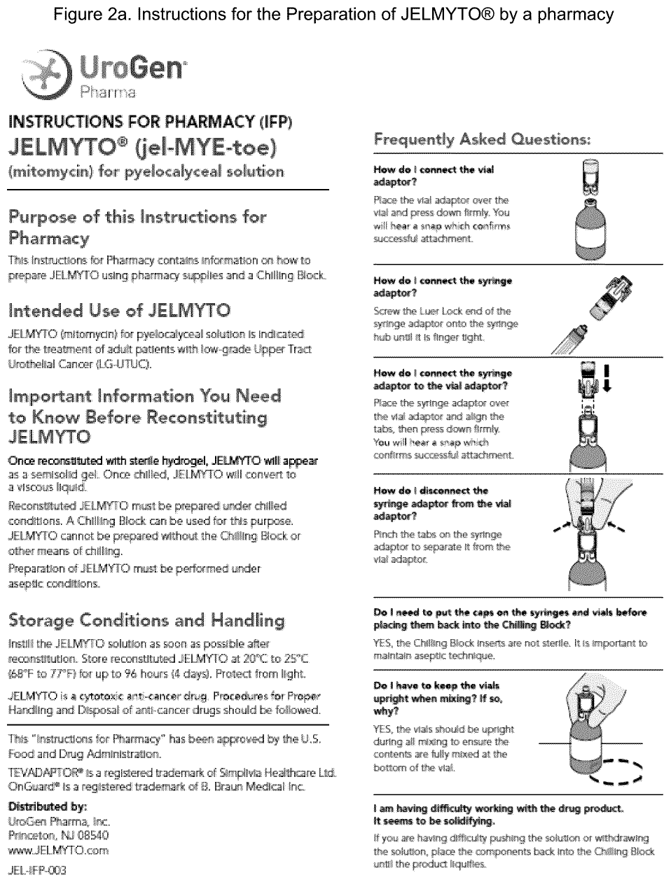

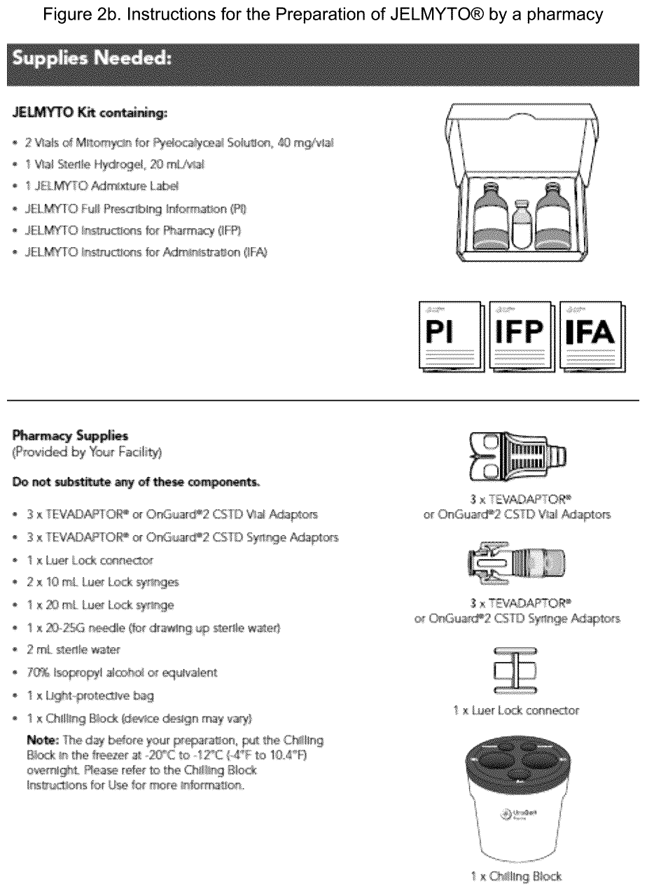

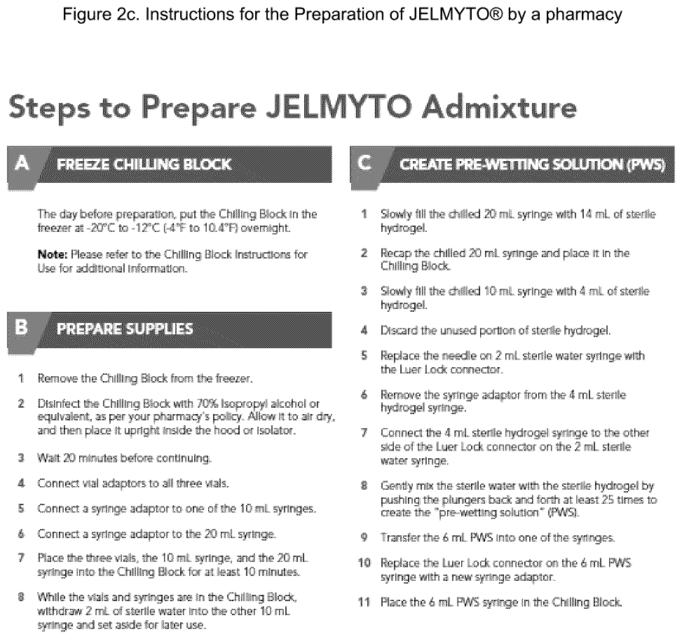

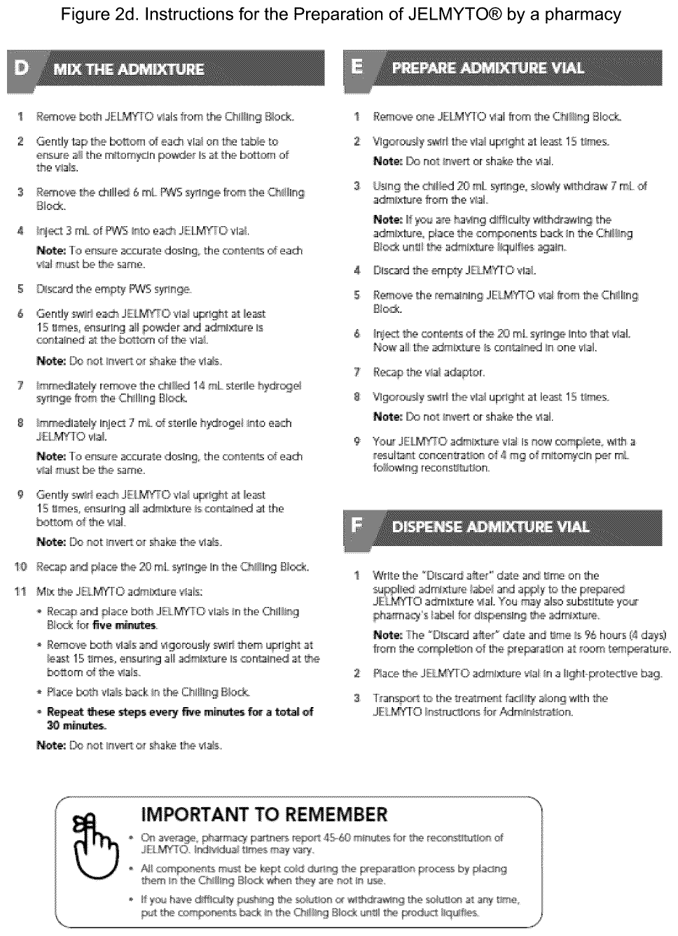

Miralles et al. evaluated the stability of Mitomycin C under different pH and temperature conditions. (MITOMYCIN C STABILITY ACCORDING TO pH AND TEMPERATURE CONDITIONS; Miralles Andreu, G; Truvols Garcia, I; Pomares Bernabeu, M; Soriano Irigaray, L; Peral Ballester, L; Navarro Ruiz A.; Hospital General Universitario de Elche, Pharmacy Department, Elche, Spain) FIG. 1, which is modified from a graph in Miralles, shows that, at room temperature, MMC was unstable (less than 90% of the initial value) in pH 4.5, 5.5, 6.0 and 7.0 solutions at various times before 24 hours.

Based on the commercial experience since launch, JELMYTO® is often administered in practice settings such as ambulatory surgery centers or physician clinics, many of which lack the ability or facilities to prepare the admixture on-site in accordance with USP 800 requirements. The current in-use holding period presents logistical challenges when pharmacies are not in close proximity to the practice, especially in remote and rural areas, since the time required to dispense the admixture from the pharmacy to the clinic exceeds the current in-use holding period. This limits access to treatment to only those practices operating within a short distance from appropriately qualified pharmacy sites.

The Applicant's unexpected discovery of a system that allows for an extended use period composition meeting all of the CQAs, was a major break-through for: (1) the pharmacy providers that prepared the compositions for administration, as well as (2) the health care providers and (3) the patients, who now would have wider access to provide and receive the needed treatments. The extension of time for use of the material increases flexibility and efficiency for health care providers and patients.

In an embodiment, a reconstituted pharmaceutical composition, comprises a thermoreversible hydrogel and a therapeutically effective amount of a therapeutic agent, wherein:
the reconstituted pharmaceutical composition is formed by combining a first composition comprising mitomycin and a second composition comprising the thermoreversible hydrogel to form the reconstituted pharmaceutical composition, and storing the reconstituted pharmaceutical composition, protected from light, at about 20° C. to about 25° C. (about 68° F. to about 77° F.) for about 8 hours to about 96 hours,
wherein the reconstituted pharmaceutical composition is formulated, and medically suitable, for administration to an internal body cavity of a subject; and
the reconstituted pharmaceutical composition is characterized by meeting the following critical quality attributes (CQAs):
   a. maintains potency of the therapeutic agent,
   b. does not produce significant quantities of degradation impurities during hold time,
   c. does not experience a material change in its appearance, pH, viscosity, and/or dissolution, and/or
   d. does not contain increased risk of microbiological exposure as indicated by a microbial challenge study and/or a microbial growth risk evaluation.

Preferably, the therapeutic agent comprises Mitomycin C and/or other Mitomycin agents, combinations thereof and the like.

In other embodiments, the reconstituted pharmaceutical composition is stored at for about 8 hours to about 96 hours; for about 8 hours to about 72 hours; for about 8 hours to about 48 hours or for about 8 hours to about 24 hours.

In some embodiments, the reconstituted pharmaceutical composition of any of the above embodiments comprises a thermoreversible hydrogel comprising:
(a) about 18% (w/w) to about 40% (w/w) ethylene oxide/propylene oxide block copolymer,
(b) about 0.05% (w/w) to about 0.5% (w/w) hydroxypropylmethylcellulose (HPMC), and
(c) water.

In other embodiments, the thermoreversible hydrogel in the reconstituted pharmaceutical composition comprises from about 20% to about 30% (w/w), preferably from about 23% to about 27% (w/w), of an ethylene oxide/propylene oxide copolymer.

In some embodiments, the ethylene oxide/propylene oxide block copolymer in the reconstituted pharmaceutical composition is a triblock copolymer.

In other embodiments, the ethylene oxide/propylene oxide block copolymer in the reconstituted pharmaceutical composition has the general formula E106 P70 E106.

In some embodiments, the thermoreversible hydrogel in the reconstituted pharmaceutical composition comprises from about 0.05% to about 0.3%, preferably from about 0.1% to about 0.3%, more preferably about 0.05% to about 0.2%, even more preferably from about 0.05% to about 0.15% HPMC, most preferably about 0.2% hydroxypropylmethylcellulose (HPMC). In other embodiments, the thermoreversible hydrogel in the reconstituted pharmaceutical composition comprises from about 0.05% to about 0.2%, preferably from about 0.1% to about 0.2%, more preferably about 0.15% to about 0.2%, even more preferably from about 0.16% hydroxypropylmethylcellulose (HPMC)

In other embodiments, the thermoreversible hydrogel in the reconstituted pharmaceutical composition comprises from about 0.4% to about 2.5%, preferably from about 0.4% to about 1.8%, more preferably about 0.5% to about 1.0%, most preferably about 0.9% to about 1′)/0 polyethylene glycol 400.

In some embodiments, the reconstituted pharmaceutical composition is prepared by combining the first composition comprising mitomycin and the second composition comprising the thermoreversible hydrogel as required by the Instructions for Pharmacy (IFP) pharmacy provided with the first composition comprising mitomycin and the second composition comprising the thermoreversible hydrogel.

In an embodiment, a kit comprises a reconstituted pharmaceutical composition of any of the embodiments described above and instructions for the administration of the pharmaceutical composition, wherein the kit does not comprise instructions inconsistent with administration to an internal body cavity of a subject, especially at time periods more than 8 hours after reconstitution.

In an embodiment of the use of the invention in treating a disease or condition affecting an internal body cavity, the method comprises administering a pharmaceutical composition as described herein to an internal body cavity of a subject after the pharmaceutical composition has been cooled to allow the admixture to turn into a liquid form suitable for administration to the internal body cavity, and the administration occurs from about 8 hours to about 96 hours following the combining of the first composition and second composition and after having been cooled to allow the admixture to turn into a liquid form suitable for administration.

In another embodiment, a method of treating a disease or condition affecting an internal body cavity, comprises administering a pharmaceutical composition to an internal body cavity of a subject, wherein:
(a) the pharmaceutical composition is formed by combining a first composition comprising a mitomycin and a second composition comprising the thermoreversible hydrogel to form an admixture, and storing the admixture, protected from light, at 20° C. to 25° C. (68° F. to about 77° F.) for about 8 hours to about 96 hours,
(b) the pharmaceutical composition is cooled to allow the admixture to turn into a liquid form for administration, wherein the cooling is for about 15 minutes to about 120 minutes, preferably about 30 minutes to about 1 hour, and
(c) the administration occurs from about 8 hours to about 96 hours following the combining of the first composition and second composition,
wherein the pharmaceutical composition is formulated, and remains medically suitable, for administration to an internal body cavity of a subject; and
the pharmaceutical composition is characterized by the following critical quality attributes (CQAs):
   a. maintains potency of the therapeutic agent,
   b. does not produce significant quantities of degradation impurities that would violate patient safety requirements during the hold time,
   c. does not experience a material change in its appearance, pH, viscosity, and dissolution, and
   d. does not contain an increased risk of microbiological exposure, as indicated by a microbial challenge study and/or a microbial growth risk evaluation.

In an embodiment, a method of using of the invention, comprises administering the pharmaceutical composition from about 8 hours to about 96 hours, about 8 hours to about 72, about 8 hours to about 48 hours, or about 8 hours to about 24 hours following the combining of the first composition and second composition followed by step c, cooling on ice for about 1 hour.

In an embodiment, the internal body cavity is an internal body cavity of the urinary tract.

In another embodiment, the internal body cavity is an upper urinary tract, urinary bladder, renal pelvis, kidney, ureter, urethra, or any combination thereof.

In other embodiments, the internal body cavity is a kidney or a urinary bladder.

In an embodiment, the disease or condition affecting an internal body cavity is a cancer.

In an embodiment, the disease or condition affecting an internal body cavity is a urinary tract cancer.

In an embodiment, the disease or condition affecting an internal body cavity is a carcinoma of the upper urinary tract, transitional cell carcinoma in the upper urinary tract, urothelial carcinoma, urothelial carcinoma of the renal pelvis and ureter, ureteral cancer, bladder cancer, renal cancer, or any combination thereof.

In an embodiment, the disease or condition affecting an internal body cavity is a bladder cancer.

In an embodiment, the internal body cavity is a kidney and the condition affecting an internal body cavity is upper urinary tract transitional cell carcinoma.

In another embodiment, the internal body cavity is a urinary bladder and the condition affecting an internal body cavity is bladder cancer.

In an embodiment, a sterile stable reconstituted pharmaceutical composition, comprises a combination and/or mixture of a first composition comprising MMC and a second composition comprising a thermoreversible hydrogel, said reconstituted pharmaceutical composition exhibiting stability for a period of more than 8 hours when stored in a sterile container, protected from light, at about 20° C. to about 30° C.

In other embodiments, the stability can be maintained for a period of up to about 100 hours, about 105 hours, about 110 hours, about 115 hours, about 120 hours, or up to about 6 weeks or more.

In another embodiment, the sterile reconstituted pharmaceutical composition can be characterized by the following quality attributes:
a. potency is maintained above 90% of MMC for more than 8 hours
b. the concentration of each of 1,2-trans-1-hydroxy-2,7-diaminomitosene and 1,2-cis-1-hydroxy-2,7-diaminomitosene is 2.5% w/w or less during the holding period of more than 8 hours and;
c. does not contain an increased risk of microbiological exposure, as indicated by a microbial challenge study and/or a microbial growth risk evaluation.

In another embodiment, the sterile reconstituted pharmaceutical composition does not experience a material change in its appearance, pH, viscosity, and/or dissolution during storage of more than 8 hours, where a material change is one that changes one or more features to a degree that makes it unsuitable for medical use and/or unable to be applied. Such a change could represent a 1° A, 2%, 3%, 4%, 5%, 6%, 7%, 8%, 9%, 10% or greater change.

In yet another embodiment, the therapeutic agent in the reconstituted pharmaceutical composition comprises Mitomycin C and the therapeutic agent is present at a concentration of about 1.33 gm/ml or about 4 mg/ml, such as a variation of about 1% to about 5%.

In still another embodiment, the reconstituted pharmaceutical composition remains stable after storage for about 8 hours to about 96 hours at about 20° C. to about 30° C.

In another embodiment, the reconstituted pharmaceutical composition can be stored for about 8 hours to about 72 hours, about 8 hours to about 48 hours or about 8 hours to about 24 hours before administration to a subject.

In a further embodiment, the thermoreversible hydrogel in a reconstituted pharmaceutical composition comprises:
(a) about 18% (w/w) to about 40% (w/w) ethylene oxide/propylene oxide block copolymer, having the general formula E101 P56 E101;
(b) about 0.05% (w/w) to about 0.5% (w/w) hydroxypropylmethylcellulose (HPMC), and
(c) water.

In another embodiment, the thermoreversible hydrogel comprises from about 20% to about 30%, from about 23% to about 27% or from about 24% to about 25% (w/w) of an ethylene oxide/propylene oxide copolymer.

In another embodiment, the reconstituted pharmaceutical composition comprises a thermoreversible hydrogel comprising about 24.1%, about 24.2%, about 24.3%, about 24.4%, about 24.5%, about 24.6%, about 24.7%, about 24.8%, or about 24.9% (w/w) of an ethylene oxide/propylene oxide copolymer.

In another embodiment, a reconstituted pharmaceutical composition comprises a thermoreversible hydrogel comprising from about 0.05% to about 0.3%, preferably from about 0.1% to about 0.3%, more preferably about 0.15% to about 0.25%, even more preferably about 0.16% to about 0.20% of hydroxypropylmethylcellulose (HPMC).

In another embodiment a reconstituted pharmaceutical composition comprises a thermoreversible hydrogel comprising from about 0.4% to about 2.5%, preferably from about 0.4% to about 1.8%, more preferably about 0.9% to about 1′)/0 polyethylene glycol 400.

In another embodiment a pharmaceutical composition is prepared by combining a first composition comprising mitomycin and a second composition comprising a thermoreversible hydrogel in accordance with Instructions for Pharmacy (IFP) provided the first composition comprises mitomycin and the second composition comprises the thermoreversible hydrogel.

In yet another embodiment, a method of extending the in-use period of a reconstituted drug product comprises combining a first composition comprising mitomycin with a second composition comprising a thermoreversible hydrogen to form a reconstituted dry product and then storing the reconstituted dry product for more than 8 hours before administration to a subject, wherein microbial contamination is limited and sterility of the reconstituted drug is maintained for more than 8 hours after said reconstitution.

In another embodiment, a kit comprises a reconstituted pharmaceutical composition as described herein and instructions for administration of the reconstituted pharmaceutical composition, wherein the kit does not comprise instructions inconsistent with administration to an internal body cavity of a subject at a time beyond 8 hours following reconstitution.

In another embodiment, a packaging system for a reconstituted drug product comprises a closed system container for storing the reconstituted formulation, wherein said system ensures stability of the product and extends its in-use period for more than 8 hours.

In another embodiment, a method of treating a disease or condition affecting an internal body cavity comprises administering a reconstituted pharmaceutical composition to an internal body cavity of a subject after the pharmaceutical composition has been cooled to allow the admixture to turn into a liquid form for administration into a body cavity, to place the composition into a liquid form suitable for administration to the internal body cavity, wherein the predetermined time can, for example be from about 15 minutes to about 2 hours; and the administration occurs from about 8 hours to about 96 hours following the combining of the first composition and second composition and after having been cooled to allow the admixture to turn into a liquid form for administration. In exemplary embodiments, a liquid form suitable for administration to an internal body cavity can be a liquid have suitable viscosity (as defined herein) and/or that can effectively carry and deliver an intended therapeutic agent such as, for example, amitomycin like mitomycin.

In still another embodiment, the internal body cavity is a cavity of the urinary tract.

In another embodiment a method of treating a disease or condition affecting an internal body cavity, wherein the internal cavity is a cavity of the urinary tract, comprises administering a reconstituted pharmaceutical composition to an internal body cavity of a subject, wherein:
(a) the reconstituted pharmaceutical composition is formed by combining a first composition comprising a mitomycin and a second composition comprising the thermoreversible hydrogel, and storing the pharmaceutical composition protected from light at 20° C. to 25° C. (68° F. to about 77° F.) for about 8 hours to about 96 hours,
(b) the reconstituted pharmaceutical composition is cooled to place the composition into a liquid form suitable for administration to the internal body cavity, wherein the predetermined time can be, for example, from about 15 minutes to about 2 hours, and
(c) the administration occurs from about 8 hours to about 96 hours following the combining of the first composition and second composition,
   wherein the reconstituted pharmaceutical composition is formulated, and medically-suitable, for administration to the internal body cavity of the subject; and
   the reconstituted pharmaceutical composition is characterized by one or more of the following quality attributes:
      a. potency is maintained above 90% of MMC for at least 8 hours
      b. the concentration of each of 1,2-trans-1-hydroxy-2,7-diaminomitosene and 1,2-cis-1-hydroxy-2,7-diaminomitosene is 2.5% w/w or less during the holding period of more than 8 hours, and
      c. does not contain an increased risk of microbiological exposure, as indicated by a microbial challenge study and/or a microbial growth risk evaluation.

In a further embodiment of a method of treating a disease or condition affecting an internal body cavity, the appearance, pH, viscosity, and/or dissolution of the reconstituted pharmaceutical composition are not materially changed. A material change is a change of a device that would make such properties unsuitable for use and/or unable to be approved. Such a change could represent a 1%, 2%, 3%, 4%, 5%, 6%, 7%, 8%, 9%, 10% or greater change in one or more of these features.

In an embodiment of a method of treating a disease or condition affecting an internal body cavity, the administration occurs from about 8 hours to about 96 hours, about 8 hours to about 72, about 8 hours to about 48 hours, or about 8 hours to about 24 hours after combining of the first composition and second composition followed by step c, cooling the combined mixture for a predetermined period of time to place the composition into a liquid form suitable for administration into an internal body cavity of a subject, wherein the predetermined time is from about 15 minutes to about s hours.

In a further embodiment of a method of treating a disease or condition affecting an internal body cavity, the internal body cavity is an internal body cavity of the urinary tract.

In yet another embodiment of a method of treating a disease or condition affecting an internal body cavity, the internal body cavity is a cavity of the upper urinary tract, urinary bladder, renal pelvis, kidney, or any combination thereof.

In still a further embodiment of a method of treating a disease or condition affecting an internal body cavity, the disease or condition affecting an internal body cavity is a cancer.

In a further embodiment of a method of treating a disease or condition affecting an internal body cavity, the disease or condition affecting an internal body cavity is a urinary tract cancer.

In yet another embodiment of a method of treating a disease or condition affecting an internal body cavity, the disease or condition affecting an internal body cavity is a carcinoma of the upper urinary tract, transitional cell carcinoma in the upper urinary tract, urothelial carcinoma, urothelial carcinoma of the renal pelvis and ureter, ureteral cancer, bladder cancer, renal cancer, or any combination thereof.

An in-use period following reconstitution can be up to about 96 hours, up to about 72 hours, up to about 48 hours, up to about 24 hours, or up to about 12 hours, combinations thereof and the like. Compositions having an extended in-use period can include, for example, compositions described in any of U.S. Pat. Nos. 9,040,074, 9,950,069, 10,039,832, 10,471,150, U.S. Patent Publication Nos. US 2020-0114008, and US 2022-0118096, and PCT Publication Number WO2011089604, each of which is hereby incorporated by reference in its entirety as if set forth herein. The compositions can be administered up to about 96 hours, up to about 72 hours, up to about 48 hours, up to about 24 hours, or up to about 12 hours following reconstitution. The compositions include pharmaceutical compositions. In some examples, the compositions can include a therapeutic agent, such as, for example Mitomycin C.

### DEFINITIONS

The term “about” means that a numerical value can have a range of ±10% of the stated value, preferably a range of ±5% of the stated value.

The term “medically suitable” means that the product maintains a combination of two or more of the following characteristics as determined by the required regulator until administration: (1) maintains potency; (2) does not form a unacceptable level of degradation products; (3) does not materially change product performance as measured by appearance, pH, viscosity, and dissolution; and (4) does not provide an increased risk of microbiological exposure as indicated by the presence of an unacceptable level of bacterial endotoxins, a microbial challenge study and a microbial growth risk evaluation. One skilled in the art would recognize that various standards have been developed to determine the acceptability of each of these characteristics. Listed below are standards for each of the characteristics.
(1) maintains potency; (USP: https://www.usp.org/sites/default/files/usp/10 document/FAQs/strength-stability-testing-compounded-preparations.pdf #:˜:text=The%2Opurpose%20of%20strength%2C%20or%20potencyV02C%20testingV020is,%C2% B110′)/025′)/02C%20or%20within%20the%2Orange%20of%2090.0° /025%E2%80%93% 20110.0%25.
(2) does not form an unacceptable level of degradation products (ICH Q3A/B, ICH M7, USP<476> and USP<1086>; See also: Establishing Impurity Acceptance Criteria As Part of Specifications for NDAs, ANDAs, and BLAs Based on Clinical Relevance (fda.gov) https://www.fda.gov/media/124859/download
(3) does not materially change product performance as measured by appearance, pH (USP<79>, viscosity (USP<911>, and dissolution (USP<911>; and
(4) does not provide an increased risk of microbiological exposure as indicated by the presence of an unacceptable level of bacterial endotoxins (USP<85>), a microbial challenge study (USP<51> and a microbial growth risk evaluation (USP<1111>).

Stable means that: (1) the potency of the reconstituted composition is maintained above 90% of MMC for more than 8 hours, and (2) the concentration of each of 1,2-trans-1-hydroxy-2,7-diaminom itosene and 1,2-cis-1 -hydroxy-2,7-diaminomitosene is 2.5% w/w or less during the holding period of more than 8 hours.

A material change, as used herein, is a change that has a negative impact on the stability of the reconstituted composition. (see also above.)

Protected from light means that the composition is placed within a darkened container, preferably a brown or dark colored container, during storage.”

The viscosity of the reconstituted composition is related to the administration of the reconstituted composition into the body.

In one embodiment, a composition of the present invention has a viscosity of less than about 1000 mPa·s at a temperature of about 4° C. to about 12° C.

In a further embodiment, a composition of the present invention has a viscosity of more than 100 Pa·s at a temperature greater than about 20° C.

According to exemplary embodiments, the material/formulation/mixture described above, additionally comprises at least one ingredient selected from:
(a) adhesive and thickening compounds;
(b) bonding agents;
(c) pH-modifying substances;
(d) diffusion coating;
(e) plasticizers;
(f) tight junction modifiers & permeability enhancers;
(g) matrix permeability increasing components;
(h) swellable excipients matrix-forming polymers;
(i) diffusion-controlled or pulsatile formulations; and,
(j) reverse thermal gelation agents.

Descriptions of components that can be used in each of these groups are provided below.
(a) adhesive and thickening compounds;

Adhesive and thickening compounds, optionally those used in the production of coated neutral pellets (e.g., consisting of sucrose, microcrystalline cellulose, citric acid) can be used. Examples are polycarbophil (polymer of acrylic acid crosslinked with divinyl glycol), hydroxypropylmethylcellulose (HPMC) and polyvinylpyrrolidone (PVP). It is likewise possible to employ other naturally, synthetic or partially synthetic polymers such as, for example methylcellulose (MC), hydroxy-propylcellulose (HPC), other hydroxyalkylcelluloses and hydroxyalkylmethylcelluloses, carboxy-methylcelluloses and salts thereof, polyacrylic acids, polymethacrylates, gelatin, starch or starch derivatives, as well as gums like guar gum and xanthan gum.
(b) bonding agents

Bonding agents that can be employed for the production of active ingredient-containing microcapsules can be used. Examples of such bonding agents are polycarbophil, cellulose, microcrystalline cellulose, cellulose derivatives (such as, for example, HMPC, HPC and low-substituted hydroxypropylcellulose (L-HPC)), dicalcium phosphate, lactose, sucrose, ethylcellulose, hydroxypropymethylcellulose acetate succinate (HPMCAS), polyvinylpyrrolidone (PVP), vinylpyrrolidone/vinyl acetate copolymer, polyethylene glycol, polyethylene oxide, polymethacrylates, polyvinyl alcohols (PVA), partially hydrolysed polyvinyl acetate (PVAc), polysaccharides (e.g. alginic acid, alginates, galactomannans), hyaluronic acid, waxes, fats and fatty acid derivatives, and any combination thereof.
(c) pH-modifying substances

The pH-modifying substances such as, for example, acids, bases and buffer substances can be into the composition with the active ingredient. The addition of these substances makes it possible to reduce markedly the pH-dependence of the release of the APIs. Examples of suitable excipients which modify the pH in the active ingredient-containing cores are: adipic acid, malic acid, L-arginine, ascorbic acid, aspartic acid, benzenesulphonic acid, benzoic acid, succinic acid, citric acid, ethanesulphonic acid, 2-hydroxyethanesulphonic acid, fumaric acid, gluconic acid, glucuronic acid, glutamic acid, potassium hydrogen tartrate, maleic acid, malonic acid, methanesulphonic acid, toluenesulphonic acid, trometamol, tartaric acid. Citric acid, succinic acid, tartaric acid, and potassium hydrogen tartrate is optionally employed.
(d) diffusion coating

Particularly suitable for producing a diffusion coating are ethylcelluloses and polymethacrylates such as, for example, EUDRAGIT® NE, EUDRAGIT® RS and RL, cellulose acetate and cellulose acetate butyrate or combinations thereof.
(e) plasticizers

Examples of plasticizers used are citric acid derivatives (e.g. triethyl citrate, tributyl citrate, acetyl triethyl citrate), phthalic acid derivatives (e.g. dimethyl phthalate, diethyl phthalate, dibutyl phthalate), benzoic acid and benzoic esters, other aromatic carboxylic esters (e.g. trimellithic esters), aliphatic dicarboxylic esters (e.g. dialkyl adipates, sebacic esters, in particular diethyl sebacate, tartaric esters), glycerol monoacetate, glycerol diacetate or glycerol triacetate, polyols (e.g. glycerol, 1,2-propanediol, polyethylene glycol of varying chain length), fatty acids and derivatives (e.g. glycerol monostearates, acetylated fatty acid glycerides, castor oil and other natural oils, Miglyol) and fatty acid alcohols (e.g. cetyl alcohol, cetylstearyl alcohol).

The nature and amount of the plasticizer are chosen so that the above-defined release according to the invention and the necessary stability of the medicinal forms is achieved. The proportion of the plasticizer is from 0% to 50%, preferably 0% to 35%, particularly preferably 0% to 25% based on the mass of the hydrogel composition.
(f) Permeability enhancers

In some embodiments, a permeability enhancer is chosen from the group consisting of anionic surfactants, non-anionic surfactants, charged polymers, dimethyl sulfoxide (DMSO), decylmethyl sulfoxide, tert-butyl cyclohexanol, fatty acids their esters and salts, ethanol, nicotinamide, urea, perfluoropolyether, monoterpene ketones, disodium citrate, succinic acid and tris.

In some embodiments, said surfactant is chosen from the group consisting of polysorbates, sodium dodecyl sulfate, and dextran sulfate.

In some embodiments, said charged polymer is chosen from the group consisting of chitosan poly-arginine, polylysine, and aliginate.

In some embodiments, the monoterpene ketone is chosen from the group consisting of (-) menthol, (-) menthone, peppermint oil, and spearmint oil.

The release rate according to exemplary embodiments is controlled by the gel composition. Certain components can increase the permeability of the admix/formulation/mixture including water-soluble polymers such as, for example, polyethylene glycols, PVP, PVA, HPMC, HPC, hydroxyethylcelluloses (HEC), MC, carboxymethylcelluloses or their salts, dextrins, maltodextrins, cylcodextrins, dextrans or other soluble substances such as, for example, urea, salts (sodium chloride, potassium chloride, ammonium chloride, etc.), sugars (sucrose, lactose, glucose, fructose, maltose etc.) and sugar alcohols (mannitol, sorbitol, xylitol, lactitol, etc.). Based on the mass of the hydrogel, the amount of the water-soluble polymers ranges from 0% to 50%, preferably 0% to 35%, particularly preferably 0% to 20%, increasing permeability components may be employed. In an exemplary embodiment, the components for increasing the permeability excludes urea.
(g) matrix permeability increasing components

The described diffusion-controlled or pulsatile formulations can be employed directly and unmodified as medicinal form. However, they may also be further processed, where appropriate with addition of excipients, to the final admix/formulation/mixture. In order to achieve a desired release profile it is also possible to combine different coated formulations in one medicinal form, and administration of an initial dose can take place for example by combination with rapid-release formulation particles, e.g. uncoated pellets, granules or powder.

In a further embodiment of the admix/formulation/mixture containing the controlled release ingredient. These so-called admix/formulation/mixture release the active ingredient by diffusion and/or erosion.

The mass ratio of active ingredient to the total mass of the admix/formulation/mixture in these novel formulations is in the range from 1:1 to 1:10,000, preferably in the range from 1:2 to 1:1,000.

The admix/formulation/mixture which can be employed are water-soluble, water-swellable or water-insoluble substances. The novel formulations preferably comprise one or more water-swellable polymers.

A preferred family of candidates to be utilized as a basis for obtaining said hydrogel is group of tri-block copolymers designated as PEG-PPG-PEG (PEG=Polyethylene glycol and PPG=Polypropylene glycol) and called Poloxamers, that produce reverse thermal gelation compositions, i.e., with the characteristic that their viscosity increases with increasing temperature up to a point from which viscosity again decreases. In particular, Poloxamer 407 possesses a gelling temperature which is above 10° C. but below the human body temperature, i.e., 37° C. This characteristic may confer the ability of a composition containing the compound to be injected or infused in liquid state into a bodily inner cavity at a low temperature and, afterwards, as the composition warms, it solidifies into a gel, thus stabilizing upon the wall of the inner body cavity.

This characteristic has allowed Poloxamer 407 (PF-127) to be used as a carrier for most routes of administration including oral, topical, intranasal, vaginal, rectal, ocular and parenteral routes.

Poloxamer 407 (PF-127) is a nonionic surfactant composed of polyoxyethylene-polyoxypropylene triblock copolymers in a concentration ranging from 20-30%. At low concentrations (10-5% to 10-4%) they form monomolecular micelles, but higher concentrations result in multimolecular aggregates consisting of a hydrophobic central core with their hydrophilic polyoxyethylene chains facing the external medium. Micellization occurs in dilute solutions of block copolymers in selected solvents above the critical micellar concentration, at a given temperature. At higher concentrations, above a critical gel concentration, the micelles can order into a lattice.

Aqueous solutions of poloxamers are stable in the presence of acids, alkalis, and metal ions. Commonly used poloxamers include the 88 (F-68 grade), 237 (F-87 grade), 338 (F-108 grade) and 407 (F-127 grade) types, which are freely soluble in water. The “F” designation refers to the flake form of the product. PF-127 has a good solubilizing capacity, low toxicity and is, therefore, considered a good medium for drug delivery systems.

PF-127 is a commercially available polyoxyethylene-polyoxypropylene triblock copolymer that possesses a general formula E106 P70 E106, with an average molar mass of about 9840-14600 (per USP). It contains approximately about 73.2±1.7% (per USP) ethylene oxide, which accounts for its hydrophilicity. It is one of the series of poloxamer ABA block copolymers. As said above, PF-127 aqueous solutions of about 20% to about 30% w/w have the interesting characteristic of reverse thermal gelation, i.e., they are liquid at refrigerated temperatures (about 4-5° C.), but gel upon warming to room temperature. The gelation is reversible upon cooling. This phenomenon, therefore, suggests that when poured onto the skin or injected into a body cavity, the gel preparation will form a solid artificial barrier and a sustained release depot.
(h) matrix-forming polymers

Water-soluble or water-swellable matrix-forming polymers preferably employed are hydroxy-propylmethylcelluloses (HPMC), hydroxyethylmethylcelluloses, hydroxypropylcelluloses (HPC), hydroxyethylcelluloses methylcelluloses (MC), ethylcelluloses, other alkylcelluloses, hydroxy-alkylcelluloses and hydroxyalkylmethylcelluloses, sodium carboxymethylcelluloses (NaCMC), alginates, galactomannans such as, for example, guar and carob flour, xanthans, polyethylene oxides, polyacrylic acids, polymethacrylic acids, polymethacrylic acid derivatives, polyvinyl alcohols (PVA), partially hydrolysed polyvinyl acetate (PVAc), polyvinylpyrrolidone (PVP), agar, pectin, gum arabic, tragacanth, gelatin, starch or starch derivatives and mixtures of these substances.

In this connection, the admix/formulation/mixture according to the invention should preferably comprise at least about 0.1-2.0% of a hydroxypropylmethylcellulose type whose nominal viscosity (measured as 2% strength aqueous solution at 20° C.) is at least about 2.7-5.0 Pa s. HPMC types preferably used have a degree of substitution of methoxy groups of about 16.5% —about 30%, particularly preferably about 19%—about 30%, and a degree of substitution of hydroxypropoxy groups of about 4%—about 32%, particularly preferably about 4%-—about 12%.

In a particularly preferred embodiment of this invention, substances which control the pH in the admix/formulation/mixture are incorporated into the admix/formulation/mixture. The addition of such pH-modifying excipients and/or the addition of substances which dissolve or are dissolved out of the admix/formulation/mixture as the pH increases, and thus increase the porosity or permeability of the admix/formulation/mixture and/or promote erosion of the admix/formulation/mixture, makes it possible to achieve a virtually pH-independent release for these preferred embodiments of the present invention.

Examples of suitable excipients which can be added to the admix/formulation/mixture according to the invention to achieve release which is as far as possible pH-independent are the following substances: adipic acid, malic acid, L-arginine, ascorbic acid, aspartic acid, benzenesulphonic acid, benzoic acid, succinic acid, cellulose phthalates, in particular cellulose acetate phthalate and hydroxypropylmethylcellulose phthalate, cellulose succinates, in particular cellulose acetate succinate and HPMCAS, citric acid, ethanesulphonic acid, 2-hydroxyethanesulphonic acid, fumaric acid, gluconic acid, glucuronic acid, glutamic acid, potassium hydrogen tartrate, maleic acid, malonic acid, methanesulphonic acid, polymethacrylates (e.g. EUDRAGIT® types), toluenesulphonic acid, trometamol, tartaric acid. Citric acid, succinic acid, tartaric acid, HPMCAS, and polymethacrylates (e.g. EUDRAGIT® L) are preferably employed. If these excipients are present in the admix/formulation/mixture according to the invention, they are typically added in a proportion of from 10 to 50% based on the total mass of the admix/formulation/mixture.

Examples of plasticizing excipients in the hydrogel formulation are propylene glycol, glycerol, triethylene glycol, butanediols, pentanols, such as pentaerythritol, hexanols, long-chain alcohols, polyethylene glycols, polypropylene glycols, polyethylene/propylene glycols, silicones, phthalic acid derivatives (e.g. dimethyl phthalate, diethyl phthalate, dibutyl phthalate), benzoic acid and benzoic esters, other aromatic carboxylic esters (e.g. trimellithic esters), citric acid derivatives (e.g. triethyl citrate, tributyl citrate, acetyl triethyl citrate), aliphatic dicarboxylic esters (e.g. dialkyl adipates, sebacic esters, in particular diethyl sebacate, tartaric esters), glycerol monoacetate, glycerol diacetate or glycerol triacetate, fatty acids and derivatives (e.g. glycerol monostearates, acetylated fatty acid glycerides, castor oil and other natural oils, Miglyol), fatty acid alcohols (e.g. cetyl alcohol, cetylstearyl alcohol), sugars, sugar alcohols and sugar derivatives (e.g. erythritol, isomalt, lactitol, mannitol, maltitol, maltodextrin, xylitol). The concentration of plasticizers is normally from 0 to 30%, preferably from 0 to 20% based on the total mass of the gel.

According to another embodiment of the present invention, at least one of the following is utilized in the admix/formulation/mixture: Poly (propylene oxide)—PPO, Poly (lactide-co-glycolic acid)—PLGA, Poly (N-isopropylacrylamide)—PNIPAM, Poly (propylene fumerate)—PPF, Poly (urethane)—PU, Poly (organophosphazene)—POP, Poloxamers of the type PEO-PPO-PEO (Poly (ethylene oxide), Poly (propylene oxide), Poly (ethylene oxide)) such as poloxamer 68, 88, 98, 108, 124, 127, 188, 237, 338 and 407, Stearic Acid, Poly (acrilic acid), Glyceryl Stearate, Cetearyl Alcohol, Sodium Stearoyl Lactylate, Hydroxy-Lenolin or any combination thereof.

We claim:

1. A sterile reconstituted pharmaceutical composition, comprising a combination and mixture of a first composition comprising mitomycin C (MMC) and a second composition comprising thermoreversible hydrogel, said reconstituted pharmaceutical composition exhibiting stability for a period of more than 8 hours when stored in a sterile container, protected from light, at about 20° C. to about 30° C.

2. The sterile reconstituted pharmaceutical composition of claim 1, characterized by the following quality attributes:
a. potency is maintained above 90% of MMC for more than 8 hours;
b. the concentration of each of 1,2-trans-1-hydroxy-2,7-diaminomitosene and 1,2-cis-1-hydroxy-2,7-diaminomitosene is 2.5% w/w or less during the holding period of more than 8 hours; and
c. the reconstituted composition meets the requirements of a microbial challenge study and/or a microbial growth risk evaluation.

3. The sterile reconstituted pharmaceutical composition of claim 1, wherein said sterile reconstituted pharmaceutical composition does not experience a material change in its appearance, pH, viscosity, and/or dissolution during storage of more than 8 hours.

4. The reconstituted pharmaceutical composition of claim 1, wherein mitomycin C is present at a concentration of about 1.33 gm/ml or about 4 mg/ml.

5. The reconstituted pharmaceutical composition of claim 1, wherein the reconstituted pharmaceutical composition remains stable after storage in a sterile container, protected from light, for about 8 hours to about 96 hours at about 20° C. to about 30° C.

6. The reconstituted pharmaceutical composition of claim 1, wherein the reconstituted pharmaceutical composition is stored for about 8 hours to about 72 hours in a sterile container, protected from light, at about 20° C. to about 30° C. before administration to a subject.

7. The reconstituted pharmaceutical composition of claim 1, wherein the reconstituted pharmaceutical composition is stored for about 8 hours to about 48 hours in a sterile container, protected from light, at about 20° C. to about 30° C. before administration to a subject.

8. The reconstituted pharmaceutical composition of claim 1, wherein the reconstituted pharmaceutical composition is stored for about 8 hours to about 24 hours in a sterile container, protected from light, at about 20° C. to about 30° C. before administration to a subject.

9. The reconstituted pharmaceutical composition of claim 1, wherein the thermoreversible hydrogel comprises:
(a) about 18% (w/w) to about 40% (w/w) ethylene oxide/propylene oxide block copolymer, having the general formula E101 P56 E101;
(b) about 0.05% (w/w) to about 0.5% (w/w) hydroxypropylmethylcellulose (HPMC), and
(c) water.

10. The reconstituted pharmaceutical composition of claim 1, wherein the thermoreversible hydrogel comprises from about 20% to about 30% (w/w) of an ethylene oxide/propylene oxide copolymer.

11. The reconstituted pharmaceutical composition of claim 1, wherein the thermoreversible hydrogel comprises from about 23% to about 27% (w/w) of an ethylene oxide/propylene oxide copolymer.

12. The reconstituted pharmaceutical composition of claim 1, wherein the thermoreversible hydrogel comprises from about 24% to about 25% (w/w) of an ethylene oxide/propylene oxide copolymer.

13. The reconstituted pharmaceutical composition of claim 1, wherein the thermoreversible hydrogel comprises an amount (w/w %) of an ethylene oxide/propylene oxide copolymer selected from the group consisting of: about 24.1%, about 24.2%, about 24.3%, about 24.4%, about 24.5%, about 24.6%, about 24.7%, about 24.8%, and about 24.9%.

14. The reconstituted pharmaceutical composition of claim 1, wherein the thermoreversible hydrogel comprises an amount (w/w %) of hydroxypropylmethylcellulose (HPMC) selected from the group consisting of: about 0.05% to about 0.3%, about 0.1% to about 0.3%, about 0.15% to about 0.25%, and about 0.16% to about 0.20%.

15. The reconstituted pharmaceutical composition of claim 1, wherein the thermoreversible hydrogel comprises an amount (w/w %) of polyethylene glycol 400 selected from the group consisting of:
about 0.4% to about 2.5%, from about 0.4% to about 1.8%, and about 0.9% to about 1%.

16. The reconstituted pharmaceutical composition of claim 1, wherein the pharmaceutical composition is prepared by combining the first composition comprising mitomycin and the second composition comprising the thermoreversible hydrogel in accordance with Instructions for Pharmacy (IFP) provided with the first composition comprising mitomycin and the second composition comprising the thermoreversible hydrogel.

17. A method of extending the in-use period of a reconstituted drug product, the method of comprising combining a first composition comprising mitomycin C with a second composition comprising a thermo reversible hydrogen to make a reconstituted dry product and then storing the reconstituted dry product for more than 8 hours before administration to a subject, wherein microbial contamination is limited and sterility of the reconstituted drug is maintained for more than 8 hours after said reconstitution.

18. A kit comprising the reconstituted pharmaceutical composition of claim 1 and instructions for administration of the reconstituted pharmaceutical composition, wherein the kit does not comprise instructions inconsistent with administration to an internal body cavity of a subject at beyond 8 hours following reconstitution.

19. (canceled)

20. A method of treating a disease or condition affecting an internal body cavity, the method comprising administering a reconstituted pharmaceutical composition of claim 1 to an internal body cavity of a subject after the pharmaceutical composition has been cooled to place the composition into a liquid form suitable for administration to the internal body cavity, wherein the predetermined time is from about 15 minutes to about 1 hour 2 hours; and the administration occurs from about 8 hours to about 96 hours following the combining of the first composition and second composition and after having been cooled to allow the admixture to turn into a liquid form for administration on ice for said predetermine time.

21. The method of claim 20, wherein the internal body cavity is a cavity of the urinary tract.

22. A method of treating a disease or condition affecting an internal body cavity, wherein the internal cavity is a cavity of the urinary tract, the method comprising administering a reconstituted pharmaceutical composition to an internal body cavity of a subject, wherein:
(a) the reconstituted pharmaceutical composition is formed by combining a first composition comprising a mitomycin and a second composition comprising the thermoreversible hydrogel, and storing the pharmaceutical composition protected from light at 20° C. to 25° C. (68 ° F. to about 77° F.) for about 8 hours to about 96 hours,
(b) the reconstituted pharmaceutical composition is cooled to place the composition into a liquid form suitable for administration to the internal body cavity, wherein the predetermined time is from about 15 minutes to about 2 hours, and
(c) the administration occurs from about 8 hours to about 96 hours following the combining of the first composition and second composition,
wherein the reconstituted pharmaceutical composition is formulated, and medically suitable, for administration to the internal body cavity of the subject; and
the reconstituted pharmaceutical composition is characterized by one or more of the following quality attributes:
a. potency is maintained above 90% of MMC for at least 8 hours,
b. the concentration of each of 1,2-trans-1-hydroxy-2,7-daminormitosene and 1,2-cis-1-hydroxy-2,7-diaminomitosene is 2.5% w/w or less during the holding period of more than 8 hours, and
c. the reconstituted composition meets the requirements of a microbial challenge study and/or a microbial growth risk evaluation.

23. The method of claim 22, wherein the appearance, pH, viscosity, and/or dissolution of the reconstituted pharmaceutical composition are not materially changed.

24. The method of claim 22, wherein the administration occurs from about 8 hours to about 96 hours after combining of the first composition and second composition followed by step c, cooling for a predetermined period of time to place the composition into a liquid form suitable for administration into an internal body cavity of a subject, wherein the predetermined time is from about 15 minutes to about 2 hours.

25. The method of claim 22, wherein the internal body cavity is an internal body cavity of the urinary tract.

26. The method of claim 22, wherein the internal body cavity is a cavity of the upper urinary tract, urinary bladder, renal pelvis, kidney, or any combination thereof.

27. The method of claim 22, wherein the disease or condition affecting an internal body cavity is a cancer.

28. The method of claim 22, wherein the disease or condition affecting an internal body cavity is a urinary tract cancer.

29. The method of claim 22, wherein the disease or condition affecting an internal body cavity is a carcinoma of the upper urinary tract, transitional cell carcinoma in the upper urinary tract, urothelial carcinoma, urothelial carcinoma of the renal pelvis and ureter, ureteral cancer, bladder cancer, renal cancer, or any combination thereof.
